(19) United States
(12) Patent Application Publication
(10) Pub. No.: US 2025/0273326 A1
(43) Pub. Date: Aug. 28, 2025

(54) HEALTHCARE RESOURCE MANAGEMENT

(71) Applicant: Axle Health Inc., Claymont, DE (US)

(72) Inventors: Adam STANSELL, Claymont, DE (US); Ian ARKO, Claymont, DE (US); Yatharth AGARWAL, Claymont, DE (US); Allen ZHU, Claymont, DE (US); Connor HAILEY, Claymont, DE (US)

(21) Appl. No.: 19/060,127

(22) Filed: Feb. 21, 2025

Related U.S. Application Data
(60) Provisional application No. 63/557,304, filed on Feb. 23, 2024.

(51) Int. Cl. G16H 40/20

(52) U.S. Cl. CPC G16H 40/20

(57) ABSTRACT

Described herein are exemplary devices, apparatuses, systems, methods, and non-transitory storage media for healthcare resource management.

### CROSS REFERENCE TO RELATED APPLICATIONS

This application claims benefit of U.S. Provisional Application No. 63/557,304, filed Feb. 23, 2024, the contents of which is incorporated herein by reference in its entirety.

### TECHNICAL FIELD

This disclosure generally relates to healthcare resource management.

### BACKGROUND

It may be desirable to optimize geographic coverage of healthcare resources, such as medical supplies and equipment, and healthcare professionals who travel and visit patients, based on the geographic distribution of demand for such resources. For example, efficient use of resources (such as professionals' time) can be optimized by scheduling resources that take advantage of patient density and minimize travel time. The healthcare professional's travel itinerary is based on factors such as patient visit times, visit locations, treatments provided to the patients, and travel conditions. However, an individual patient, who lacks complete knowledge of these factors, would not know how to schedule an appointment that would reduce the travel time. An assistant may help schedule the appointments. However, due to the variability of these factors in real time and limited insight to demand and available resources, the assistant may not necessarily schedule appointments that can help reduce the travel time. In some cases, the patient can provide a large window of time (e.g., tomorrow during business hours), and the healthcare professional can visit during the best time during the window. However, the patient may spend significant time waiting, and the healthcare professional may not visit at a time that optimizes travel time.

As a result, the healthcare professional's time is often wasted due to inefficient traveling, delaying the patient's access to care. Similarly, suboptimal scheduling can compromise the efficacy of some medical treatments, or prevent their use altogether, such as where such treatments rely on certain medical supplies (e.g., infusion chemicals) with limited shelf life or that require expensive or time-sensitive handling (such as refrigeration) during transport. Because such treatments are provided by healthcare professionals, the two problems are linked.

### SUMMARY

Described herein are exemplary devices, apparatuses, systems, methods, and non-transitory storage media for care provider visit management. The disclosed devices, apparatuses, systems, methods, and non-transitory storage media allow the efficiency of traveling healthcare professionals and other medical resources to be optimized based on the geographic distribution of demand.

The coverage may be optimized by providing available visit times to a patient determined based on the healthcare professional's dynamic reachability, which may be determined based on the healthcare professional's itinerary. For example, the dynamic reachability can express an optimal distance of travel based on a demand density of an area of the healthcare professional. Using the dynamic reachability, optimal route and optimal appointment time options for future visits are determined based on a number of booked visits (e.g., zero, one, two, more than two). In response to an addition of a booked visit, the healthcare professional's route is optimized to minimize travel time, allowing the patient to access care more promptly and earlier compared to existing methods of appointment scheduling.

Embodiments of the present disclosure provide numerous technical advantages. For example, the disclosed systems and methods allow the healthcare professional's time to be maximized for providing care and minimized for traveling. As a result, the patient may receive care earlier and more efficiently. Similarly, patients may have expanded access to time-sensitive medical treatments, or treatments that rely on time-sensitive resources (e.g., infusion medicine whose potency decreases over time). Because healthcare professionals deliver time-sensitive medical treatments, which may themselves rely on time-sensitive resources (e.g., infusion medicine whose potency decreases over time), optimizing travel routes and times for healthcare professionals can address the problem of limited patient access to valuable medical treatments.

As another example, the disclosed systems and methods would allow available visit times, determined to optimize the healthcare professional's time, to be displayed for patient selection, reducing the patient's wait time because the available appointments are more definite and not a larger window of time (e.g., tomorrow during business hours). The available visit times can be presented on a graphical user interface for patient selection, simplifying the visit booking process for the patient (e.g., without a need to coordinate with an assistant or carve out a large window of time to wait for the visit), simplifying the itinerary planning process for the healthcare professional (e.g., without a need to determine an itinerary that would optimize visit times), and reducing cognitive load for the patient and the healthcare professional.

As another example, the disclosed systems and methods dynamically determine available visit times as conditions associated with the healthcare professional (e.g., patient visit times, visit locations, treatments provided to the patients, travel conditions, calendar information) change in real time. Due to the time sensitivity of these conditions, an assistant may not react to these variations in time to determine and communicate optimized available appointment time to patients. As another example, because the healthcare professional's time is more optimized, equipment used for care can be used more efficiently, allowing more costly and less available equipment to treat more patients. As another example, because the available visit times can be determined via one platform, the disclosed systems and methods allow the patient to schedule visits with healthcare professionals from different providers, improving the patient's access to care.

As a result of these examples, visit times are more efficiently determined to optimize healthcare professional travel time, reduce patient wait time, and improve patient access to care in a faster and less complex manner while consuming less device power. That is, determination and selection of optimal available visit times are performed by one system with less manual inputs required, in contrast to coordination between multiple users and their devices.

In some embodiments, a method comprises: determining an itinerary associated with a healthcare resource, determining, based on the itinerary, a number of booked visits associated with a healthcare resource, determining, based on the itinerary, slack associated with the healthcare resource, determining, based on the slack, one or more available visit times for the healthcare resource, and causing display of a graphical user interface comprising the one or more available visit times.

In some embodiments, a non-transitory computer-readable medium stores instructions that, when executed by one or more processors, cause the one or more processors to perform the above method.

In some embodiments, a system comprises: a display, and one or more processors configured to perform the above method.

Although examples of the disclosure are described with respect to managing healthcare resources, it should be appreciated that the disclosed systems and methods may be used for managing and optimizing other kinds of resources. It should be appreciated that healthcare resources can be healthcare professionals, medical resources, and/or other resources related to healthcare. It should also be appreciated that examples described with respect to a healthcare professional may be used for managing and optimizing other kinds of healthcare resources.

The embodiments disclosed are only examples, and the scope of this disclosure is not limited to them. Particular embodiments may include all, some, or none of the components, elements, features, functions, operations, or steps of the embodiments disclosed above. Embodiments according to the invention are in particular disclosed in the attached claims directed to a method, a storage medium, a system, and a computer program product, wherein any feature mentioned in one claim category, e.g., method, can be claimed in another claim category, e.g., system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 illustrates exemplary demand, in accordance with some embodiments.

FIGS. 2A and 2B illustrate exemplary representations associated with a booked visit and a potentially available visit for optimizing a healthcare professional's itinerary, in accordance with some embodiments.

FIG. 3 illustrates representations associated with a booked visit and a potentially available visit for optimizing a healthcare professional's itinerary, in accordance with some embodiments.

FIG. 4 illustrates representations associated with a booked visit and a potentially available visit for optimizing a healthcare professional's itinerary, in accordance with some embodiments.

FIG. 5 illustrates representations associated with a booked visit and potentially available visits for optimizing a healthcare professional's itinerary, in accordance with some embodiments.

FIGS. 6A and 6B illustrate representations associated with booked visits and a potentially available visit for optimizing a healthcare professional's itinerary, in accordance with some embodiments.

FIG. 7 illustrates representations associated with booked visits and a potentially available visit for optimizing a healthcare professional's itinerary, in accordance with some embodiments.

FIG. 8 illustrates representations associated with booked visits for optimizing a healthcare professional's itinerary, in accordance with some embodiments.

FIG. 9 illustrates representations associated with a booked visit for optimizing a healthcare professional's itinerary, in accordance with some embodiments.

FIGS. 10A-10C illustrate representations associated with booked visits for optimizing a healthcare professional's itinerary, in accordance with some embodiments.

FIG. 11 illustrates representations associated with booked visits and a potentially available visit for optimizing a healthcare professional's itinerary, in accordance with some embodiments.

FIG. 12 illustrates representations associated with booked visits for optimizing a healthcare professional's itinerary, in accordance with some embodiments.

FIG. 13 illustrates representations associated with booked visits for optimizing a healthcare professional's itinerary, in accordance with some embodiments.

FIG. 14 illustrates exemplary demand, in accordance with some embodiments.

FIG. 15 illustrates representations associated with a booked visit for optimizing a healthcare professional's itinerary, in accordance with some embodiments.

FIG. 16 illustrates representations associated with booked visits for optimizing a healthcare professional's itinerary, in accordance with some embodiments.

FIGS. 17A and 17B illustrate representations associated with booked visits and a potentially available visit for optimizing a healthcare professional's itinerary, in accordance with some embodiments.

FIG. 18 illustrates an exemplary method for care provider visit management, in accordance with some embodiments.

FIG. 19 illustrates an exemplary system, in accordance with some embodiments.

### DETAILED DESCRIPTION

In the following description of embodiments, reference is made to the accompanying drawings which form a part hereof, and in which it is shown by way of illustration specific embodiments which can be practiced. It is to be understood that other embodiments can be used, and structural changes can be made without departing from the scope of the disclosed embodiments.

Described herein are exemplary devices, apparatuses, systems, methods, and non-transitory storage media for care provider visit management. The disclosed devices, apparatuses, systems, methods, and non-transitory storage media allow the efficiency of traveling healthcare professionals and other medical resources to be optimized based on the geographic distribution of demand.

The coverage may be optimized by providing available visit times to a patient determined based on the healthcare professional's dynamic reachability, which may be determined based on the healthcare professional's itinerary. For example, the dynamic reachability can express an optimal distance of travel based on a demand density of an area of the healthcare professional. Using the dynamic reachability, optimal route and optimal appointment time options for future visits are determined based on a number of booked visits (e.g., zero, one, two, more than two). In response to an addition of a booked visit, the healthcare professional's route is optimized to minimize travel time, allowing the patient to access care more promptly and earlier compared to existing methods of appointment scheduling.

As described in more detail herein, in some embodiments, the disclosed system is configured to receive data about the healthcare services required for an in-home clinical visit, and output a set of available visit time slots (which are optimized, as described herein) that a patient can book for their visit. The available visit time slots are determined such that, with reasonable certainty, all visits for all patients would fit within a set of custom optimization constraints once all visits have been booked.

For example, the system can predict the density of demand for different types of visits within a geographical area around the patient (who may be looking to book a visit). Then using the demand density to predict a visit distribution and a model to predict travel time between different points, the system can calculate efficiency of a route associated with each possible time slot. The system can filter out any time slots where the predicted route would violate one or more optimization constraints and provide (e.g., for display on a graphical user interface) only time slots that are predicted to meet the optimization constraints. Patients can then choose (e.g., via the graphical user interface) from any of the optimized time slots offered to them, and once patients have booked their visits, each route for each healthcare professional would have a predicted efficiency that can meet the optimization constraints. In some embodiments, the route is updated dynamically (e.g., when parameters for optimizing available visit time slots change, when a new visit is booked, when a visit is cancelled).

Embodiments of the present disclosure provide numerous technical advantages. For example, the disclosed systems and methods allow the healthcare professional's time to be maximized for providing care and minimized for traveling. As a result, the patient may receive care earlier and more efficiently. Similarly, patients may have expanded access to time-sensitive medical treatments, or treatments that rely on time-sensitive resources (e.g., infusion medicine whose potency decreases over time). Because healthcare professionals deliver time-sensitive medical treatments, which may themselves rely on time-sensitive resources (e.g., infusion medicine whose potency decreases over time), optimizing travel routes and times for healthcare professionals can address the problem of limited patient access to valuable medical treatments.

As another example, the disclosed systems and methods would allow available visit times, determined to optimize the healthcare professional's time, to be displayed for patient selection, reducing the patient's wait time because the available appointments are more definite and not a larger window of time (e.g., tomorrow during business hours). The available visit times can be presented on a graphical user interface for patient selection, simplifying the visit booking process for the patient (e.g., without a need to coordinate with an assistant or carve out a large window of time to wait for the visit), simplifying the itinerary planning process for the healthcare professional (e.g., without a need to determine an itinerary that would optimize visit times), and reducing cognitive load for the patient and the healthcare professional.

As another example, the disclosed systems and methods dynamically determine available visit times as conditions associated with the healthcare professional (e.g., patient visit times, visit locations, treatments provided to the patients, travel conditions, calendar information) change in real time. Due to the time sensitivity of these conditions, an assistant may not react to these variations in time to determine and communicate optimized available appointment time to patients. As another example, because the healthcare professional's time is more optimized, equipment used for care can be used more efficiently, allowing more costly and less available equipment to treat more patients. As another example, because the available visit times can be determined via one platform, the disclosed systems and methods allow the patient to schedule visits with healthcare professionals from different providers, improving the patient's access to care.

As a result of these examples, visit times are more efficiently determined to optimize healthcare professional travel time, reduce patient wait time, and improve patient access to care in a faster and less complex manner while consuming less device power. That is, determination and selection of optimal available visit times are performed by one system with less manual inputs required, in contrast to coordination between multiple users and their devices.

Although examples of the disclosure are described with respect to managing healthcare resources, it should be appreciated that the disclosed systems and methods may be used for managing and optimizing other kinds of resources. It should be appreciated that healthcare resources can be healthcare professionals, medical resources, and/or other resources related to healthcare. It should also be appreciated that examples described with respect to a healthcare professional may be used for managing and optimizing other kinds of healthcare resources.

In addition, although examples of the disclosure are represented geometrically, it should be appreciated that the geometric representations are illustrated for describing the disclosed optimizations and are not meant to be limiting.

In some embodiments, the system may receive one or more of visit characteristics, optimization constraints, and predicted travel times. The system can determine a set of available time slots that would meet the constraints, in view of the visit characteristics and predicted travel times. The system can then present the set of available time slots to a patient looking to book a visit on a graphical user interface for the patient to select. After selection of an available time slot via the graphical user interface, a visit at the selected time slot is booked. The system may use information associated with the booked visit for future available visit time slot optimization. The system advantageously allows visits for all patients to meet the optimization constraints, even when visits are booked sequentially, and patients may not have knowledge of any other visits. Examples of available visit determination are now described.

As mentioned above, the coverage by healthcare professionals may be balanced by providing available visit times to a patient determined based on the healthcare professional's dynamic reachability, which may be determined based on a healthcare professional's itinerary. For example, dynamic reachability may indicate an optimal travel distance related to density of a market. This may be determined by setting a maximum continuous travel duration. Using this method, the system can consider how to optimally route a future visit (e.g., an available visit that can be presented to a patient) based on a number of booked visits (e.g., zero, one, two or more). As a subsequent visit is added, the system determines how to route the next visit based on an existing amount of booked visits.

The maximum continuous travel duration may be furthest a healthcare professional can travel between any two visits. If the healthcare professional needs to travel further than the maximum continuous travel duration, then there is a potential for available visits between the two visits (e.g., there is a demand in between the two visits within the healthcare professional's projected travel time), such that the healthcare professional's time is not wasted traveling for the long visit. The system would build in time, as described herein, for these potential visits, which may be called a buffer. The total amount of time built in for these potential available visits may be called slack. Buffer and slack used to optimize the healthcare professional's time are described in more detail herein.

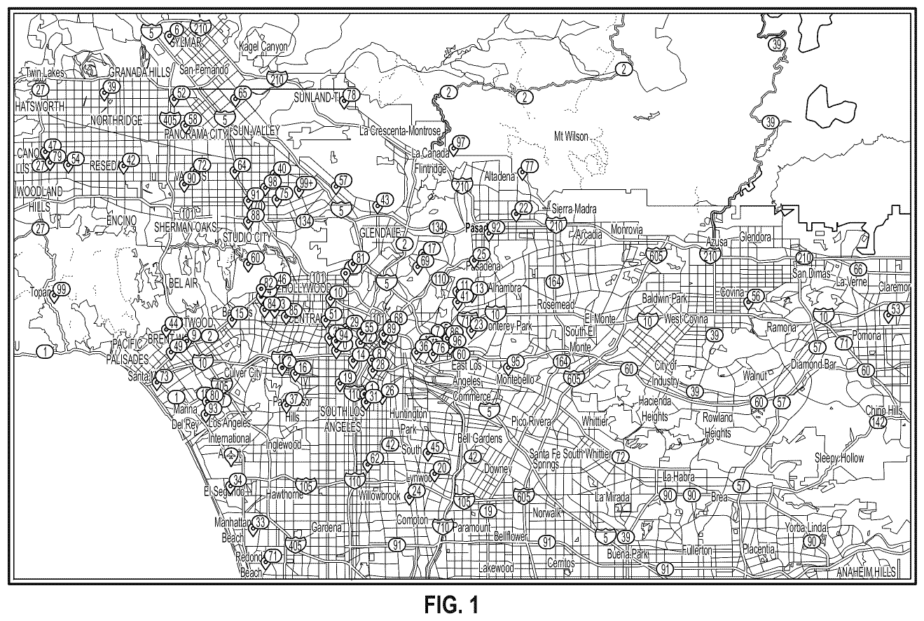

Because the maximum continuous travel duration can be the furthest a healthcare professional can travel between any two visits, the maximum continuous travel duration would be optimal if the average of travel durations between each location of projected demand to its nearest neighboring location. That is, the maximum continuous travel duration would be optimal if it is an average travel time between adjacent areas of demand, such that the healthcare professional would not miss an area of demand while traveling to a next visit. FIG. 1 illustrates exemplary demand, in accordance with some embodiments. More specifically, FIG. 1 illustrates distribution of demand selected from a random week. The areas of demand may be indicated by each pin, and the number on each pin may indicate a level of demand for each corresponding area.

In some embodiments, the system determines that a number of booked visits is zero. In these instances, the probability of visit may be based on the distribution of demand. For example, the probability of the visit would be higher for an area with higher demand. In some embodiments, in addition to the distribution of demand, the probability of the visit is determined based on other factors, such as user (e.g., patient, healthcare professional, provider) inputs.

In some embodiments, the system determines that a number of booked visits is one. In some embodiments, an amount of time the system determines for one visit to another is proportional to a total travel duration between a booked visit and an available visit.

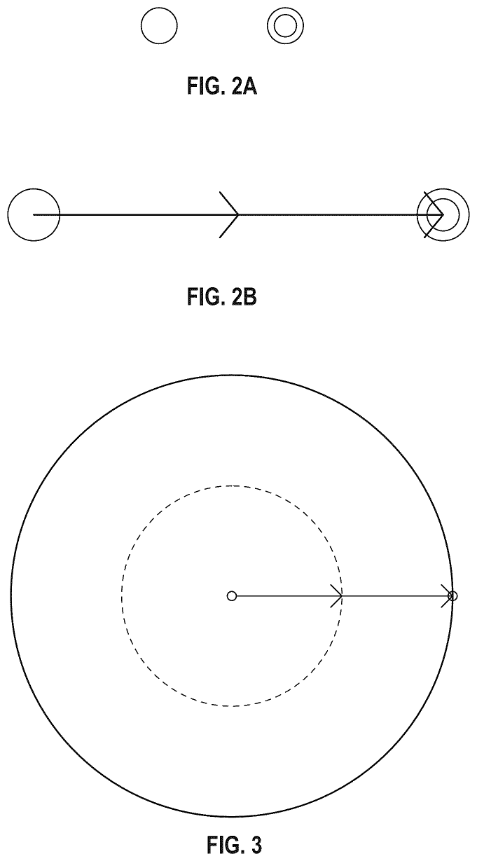

FIGS. 2A and 2B illustrate exemplary representations associated with a booked visit and a potentially available visit for optimizing a healthcare professional's itinerary, in accordance with some embodiments. As illustrated in these Figures, the single circle (on the left) represents a booked visit, and circles (on the right) represents a potential available visit (which the system may use to construct availability presented to a patient, which may be a new visit requested by a patient). Each of the two arrows in FIG. 2B and its length represent a maximum continuous travel duration. So, in this example, the booked visit and the potential available visit are two maximum continuous travel durations apart.

In some embodiments, the system determines the maximum continuous travel duration based on random sampling and calculations of nearest neighbors for a set of addresses. In some embodiments, the system determines the maximum continuous travel duration based on demands associated with areas (e.g., different zip codes). In some embodiments, the system determines the maximum continuous travel duration based on observed population dynamics of a market of the healthcare professional.

As an example, the travel duration between a booked visit and a potential available visit is 25 minutes, and the maximum continuous travel duration is 30 minutes. Because the example travel duration is 25 minutes, the potential available visit would not be less than 25 minutes since it would not be possible for the healthcare professional to reach this potential next visit. This idea can be referred to as reachability. Visits that would take a particular amount of distance or time from one to another, and the scheduling of these visits may need to abide by the healthcare professional's ability to reach from one visit to the next.

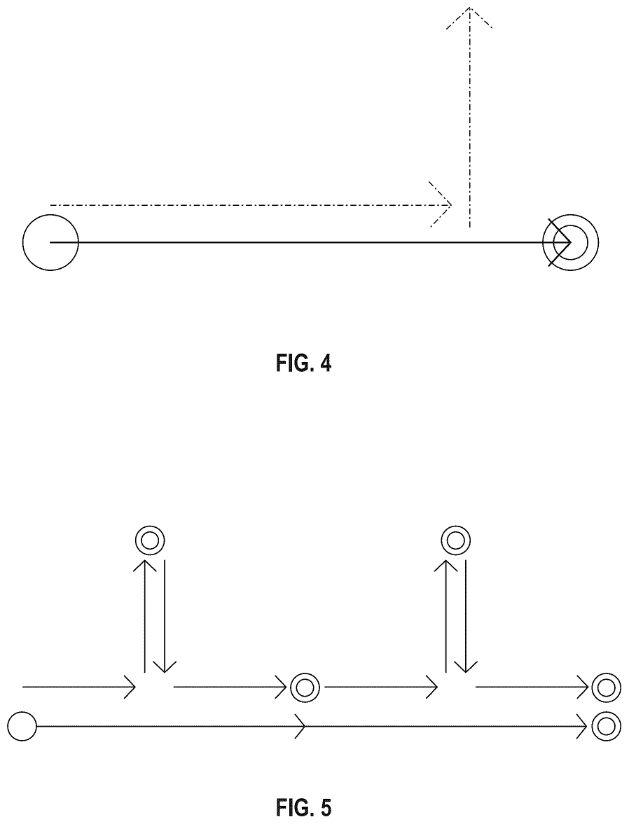

In some examples, the travel duration may be greater than the maximum continuous travel duration, which would leave space between two visits (e.g., potential to add additional visits between these two visits and reduce travel between visits). In some embodiments, the maximum continuous travel duration can be represented as a radius (with length corresponding to a maximum continuous travel duration), as illustrated in FIG. 3. The inner circle may represent the radius associated with the first maximum continuous travel duration, and the outer circle may represent the radius associated with the second maximum continuous travel duration. Each step taken away from the booked visit, a buffered visit worth of time may be included.

In the illustrated representations, it should be appreciated that the visit duration part of a buffer is not shown for better clarity.

In some embodiments, the total travel duration between a booked visit and a potential available visit can be divided by the maximum continuous travel duration, which would determine the number of buffers and slack.
NumBuffers=TravelDuration/MaxTravelDuration
Slack=NumBuffers*BufferDuration

For example, the above relationship may be used to determine buffers and slack when the travel duration exceeds the maximum continuous travel duration.

As illustrated in FIG. 4, if the buffer duration only includes a visit duration worth of time, then it would not be possible to book a visit any distance off route between the booked visit and the potential available visit. For example, if the healthcare professional were to travel further than 15 minutes off the route between the booked visit and the potential available visit, then the maximum continuous travel duration (represented by horizontal arrow) would be violated. FIG. 5 illustrates a representation of a more stable itinerary for the booked visit and the potential available visit.

For this reason, some additional travel time would be added into the buffered duration, as illustrated in FIG. 5. As shown in the Figure, the booked visit and the first potential available visit are two maximum continuous travel durations apart, and buffers associated with additional potential available visits can be included, achieving the benefits described herein. For example, for one part spent traveling on a route, there is another part allocated to traveling to a next visit. Each set of arrows between adjacent potential available visits in FIG. 5 may represent a buffer and the length of one set of arrows may represent slack associated with a buffer.

In some embodiments, as shown in FIG. 5, the travel component of the buffer duration (e.g., without the visit duration) may be equal to the maximum continuous travel duration. It may not be necessary to add the underlying travel duration between the booked visit and the first potential available visit into slack. In addition to the buffered time, one additional maximum continuous travel duration worth of time is added to the slack, to account for a travel duration between the last buffered slot (e.g., the slot adjacent to the left of the first potential available visit) to the first potential available visit. In view of these considerations, in some embodiments, the number of buffers and slack are determined as follows.
BufferDuration=Visit Duration+MaxTravelDuration
NumBuffers=((2*TravelDuration)/MaxTravelDuration)−1
Slack=(NumBuffers*BufferDuration)+MaxTravelDuration

It should be appreciated that the parameters for determining slack and optimizing available visit times may not be consistent and may be updated. For example, the system may receive updated information (e.g., updated patient visit times, updated visit locations, updated treatments provided to the patients, updated travel conditions, updated calendar information) regarding the healthcare professional's itinerary, and the parameters are updated accordingly for updated calculations (e.g., of slack).

In some embodiments, NumBuffers is a whole number, such that:
NumBuffers=floor[((2*TravelDuration)/MaxTravelDuration)−1]

By determining the number of buffers based on the above relationship, a potential for fractional visit can be eliminated. A fractional visit may cost a provider money and resources for an appointment that may not be fulfilled due to insufficient time.

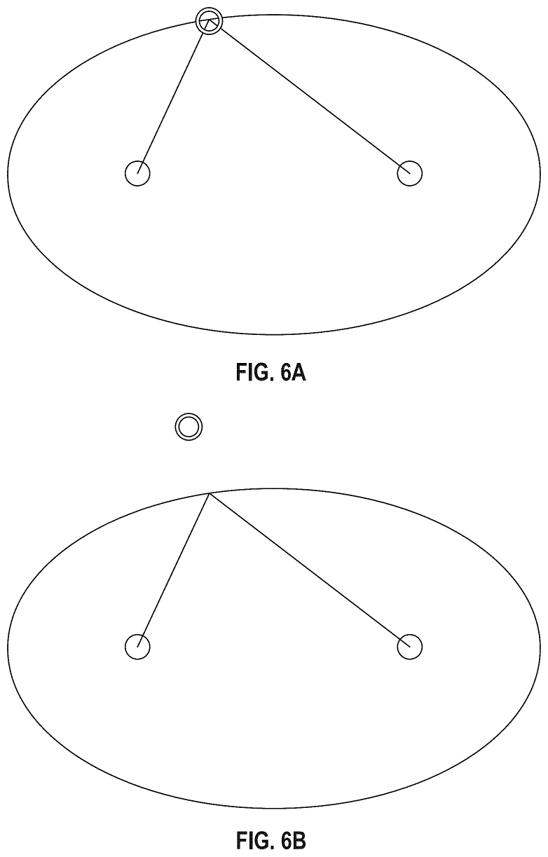

In some embodiments, the system determines that a number of booked visits is two. A scenario with two booked visits may be represented by a string and two pins, as illustrated in FIG. 6A. The two pins may be the two booked visits having a fixed travel duration between them (e.g., the linear distance between the two booked visit in the representation). If slack is added, then the string would be longer than the linear distance between the two booked visits. In some embodiments, the slack can be determined according to the above discussions. Because slack includes one or more buffers, it may be desirable to schedule visits between the two booked visits more efficiently by optimizing the number of visits between the two booked visits.

If the two booked visits were connected by a thread representing the slack, an ellipse can be defined by the further point in the thread, as illustrated in FIG. 6A. That is, the boundary of the ellipse (e.g., reachability frontier) may define the furthest possible available visit between the two booked visits for the given slack. The ellipse can represent the amount of slack and how the slack can be utilized, which can be called tautness. FIG. 6B illustrates a scenario when a visit is requested outside of the ellipse, meaning there is not enough time in the slack to accommodate the visit while respecting the times of the two booked visits.

For example, if the total slack between two booked visits includes one buffer, then the tautness would be optimized because the total slack has been utilized. However, if for example the total slack between the two booked visits includes five buffers and if a visit on the boundary of the ellipse is booked, then the slack may not be optimally utilized because all the slack (e.g., the five buffers) is used for one visit, but can be used for more (e.g., can be used for five visits instead).

Therefore, more slack may be consumed for appointments closer to the ellipse boundary than appointments farther away from the boundary. To optimize the use of slack in the scenario with two booked visits, the available visit times are determined according to this idea. Therefore, more visits may be available closer to the middle of the ellipse than farther away from the ellipse, to improve utilization of slack.

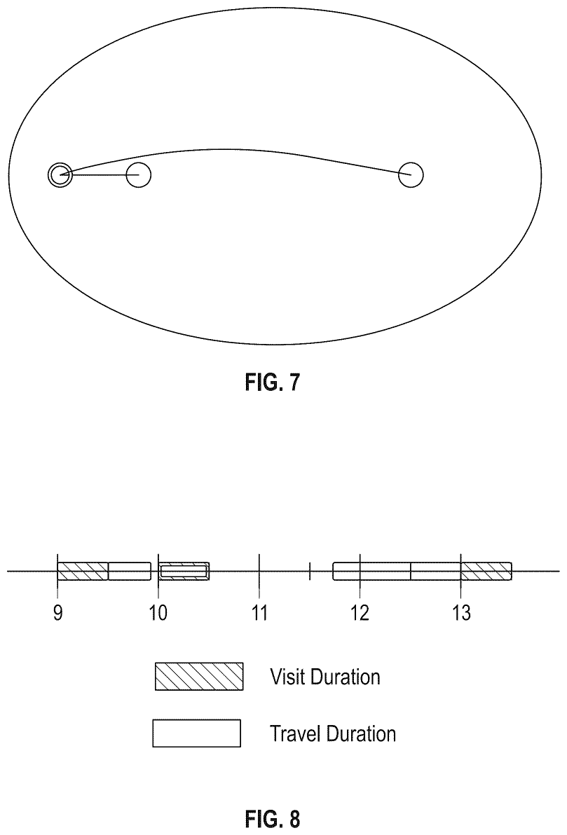

For example, a rule stating that a requested visit should not take more than an average of one buffered slot can be enforced to optimize the use of slack. This rule can be called the “N−1 rule.” As an example, a potential available visit is in a direction opposite to a direction from the left booked visit to the right booked visit (as illustrated in FIG. 7), the left booked visit is at 9:00, and the right visit is booked at 13:00 according to slack (based on 30 minute maximum continuous travel duration, two max travel duration times apart, and 30 minute travel durations).

This example is represented in FIG. 8, where the booked visit durations are represented in a shaded pattern and the travel duration for traveling from the first visit and traveling to the second visit are represented in a non-shaded pattern. The box with a shade boundary may represent travel time associated with the potential available visit represented in FIG. 7. As illustrated in this example, there may be too much time available for one visit (e.g., there is two hours of slack between 9:50 and 11:50), and slack is not efficiently utilized. Thus, in some embodiments, additional constraints may be considered to better enforce the N−1 rule.

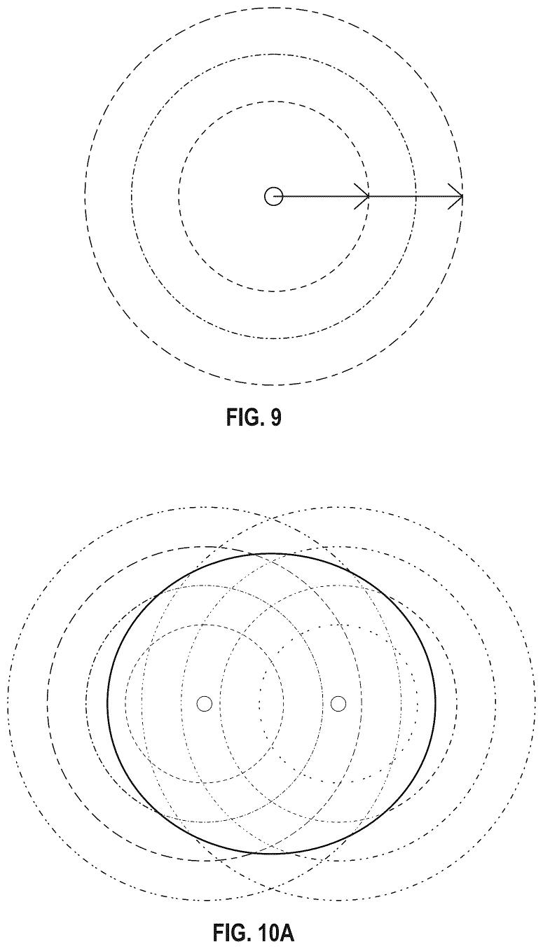

In some embodiments, the travel duration between the two booked visits and the potential available visits is buffered. The determination may be represented by a buffer level set diagram, as illustrated in FIG. 9. In this Figure, each ring may represent how many buffers would remain based on the travel duration from a booked visit to an arborary point inside a ring.

In the example in this Figure, the middle ring (smallest ring in the middle) would represent zero buffered visits because it has less than one maximum continuous travel duration (e.g., length of one arrow). The second ring (one size bigger than the middle ring) would include one buffer slot because it is 1 to 1.5 travel durations away from the booked visit. That is, floor (2*1)−1=1, and floor (2*1.5)−1=2.

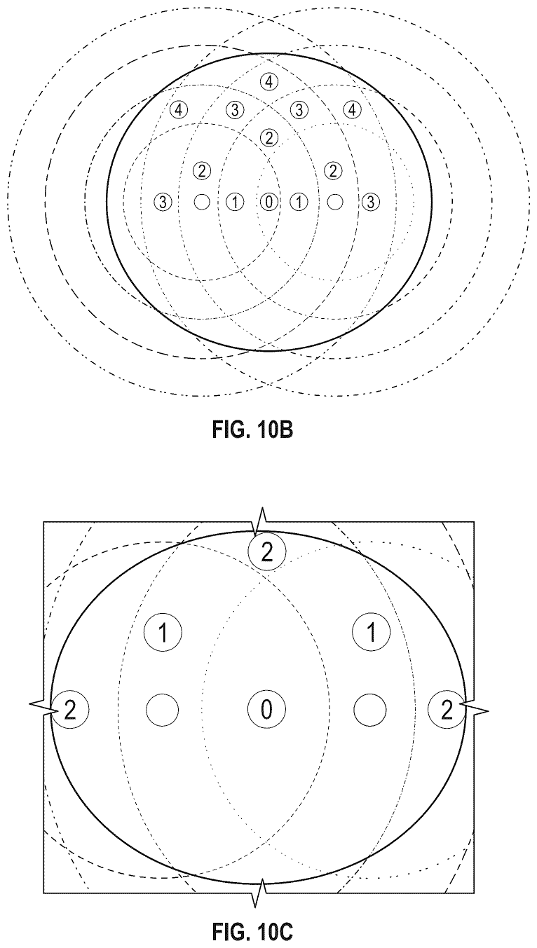

If the buffer were determined based on travel durations, there would be two travel durations-the duration from the left booked visit to a potential available visit and the duration from right booked visit and the potential available visit. To leave enough space to fit visits on both the left and right of the potential available visit, the system would buffer based on travel duration for both the left and right travel durations. This can be represented in FIG. 10A.

As illustrated in FIG. 10A, the two booked visits and their buffered level set diagrams can be represented as overlapping. An ellipse representing slack associated with the two booked visits is also illustrated in the Figure. The sum of total buffers from the left and right may indicate whether a potential available visit would meet one or more constraints for optimizing the healthcare professional's itinerary. As illustrated, the number of buffers would increase as a potential available visit (not shown) moves further away from the center between the two booked visits. The area near the left visit is buffered more heavily from the right, opening up more time slots closer to the left visit; the reverse may be true.

FIG. 10B shows an annotated version of the representations in FIG. 10A. More specifically, the annotations are a total buffer numbers that the system would leave for different areas of the representations. From this Figure, it can be seen that along the optimal route (e.g., a direct route between the two booked visits), there would be less buffers. As a point on the diagram (not shown) moves from the left booked visit to the right booked visit, the one buffer goes from being on the right to being on the left. That is, if there is two time slots worth of time in the slack, then there should be one visit worth of space nearer to the left booked visit and one visit worth of space nearer to the right booked visit. It can also be seen that as a point in the diagram (not shown) moves closer and closer to the ellipse boundary, the system would leave more and more buffers.

FIG. 10C shows an annotated representation of two booked visits having less slack, compared to the example described with respect to FIG. 10B. In this example, the two booked visits are closer together than the two visits in the example described with respect to FIG. 10B. In this example, it would also be possible to book additional visits. There is one buffered slot. The system is leaving one buffer to the left and right when near the left and right booked visits, respectively. In addition to the buffers, the slack also includes one additional maximum continuous travel duration, making an additional visit possible. When consuming slack, the system allocates buffers based on the travel duration, and because one buffer worth of space to the left or right is allocated, there is open time for booking an additional appointment.

In some examples, if the two booked visits are sufficiently close and there is enough time for a remaining visit, the itinerary can be optimized better than expected using this methodology, compare to merely using the maximum continuous travel duration, even when a buffer is recorded.

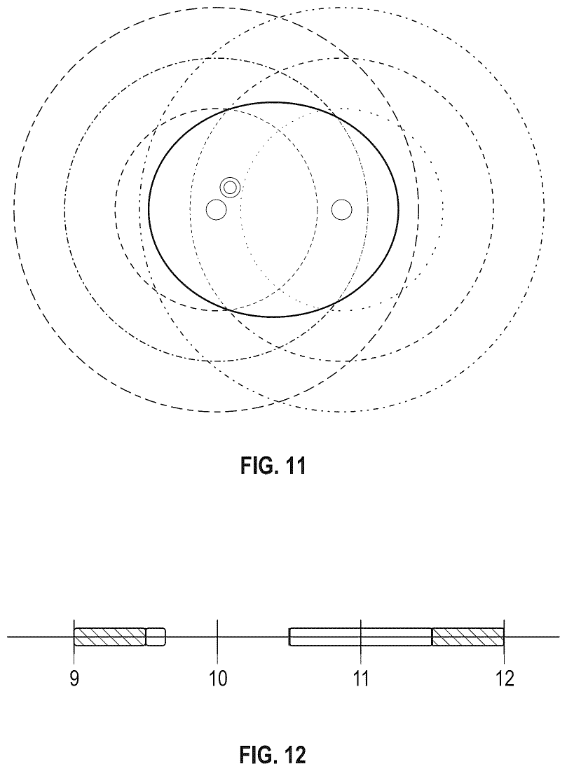

FIG. 11 illustrates representations associated with booked visits and a potentially available visit for optimizing a healthcare professional's itinerary, in accordance with some embodiments. The potential available visit is represented by the single circle and added to the representation of the example in FIG. 10C to illustrate a determined potential available visit. FIG. 12 illustrates a timeline representation of this example, showing that the healthcare professional's time is better optimized (compared to the example illustrated in FIG. 8) for one potential visit between the two booked visits.

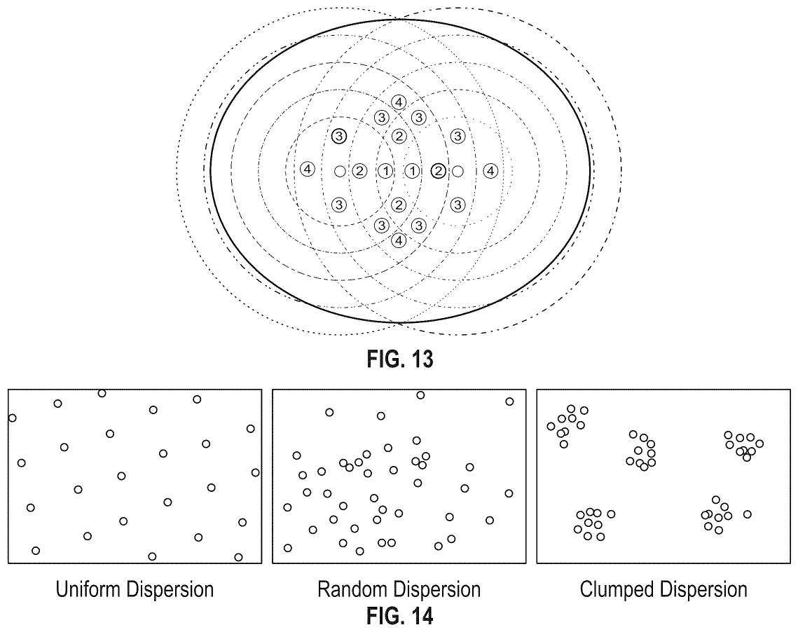

FIG. 13 shows an annotated representation of two booked visits having more slack, compared to the example described with respect to FIG. 10B. In this example, the two booked visits are farther away from each other than the two visits in the example described with respect to FIG. 10B. As indicated by the annotations, even though this example yields a larger ellipse, the bookable space is bounded by the two booked visits.

In some embodiments, the system determines that a number of booked visits is more than two. If the number of booked visits is more than two, then the booked visits may be broken into groups of two, and the available visits associated with each group of two can be determined according to the two visit examples described above. For example, for three visits a, b, and c, a first set of available visits can be determined for visits a and b, and a second set of available visits can be determined for visits c and b.

Thus, the described methods for optimizing the healthcare professional's itinerary allow appointment booking to be more scalable, since the determination of available visit times can be broken down into smaller determinations. Furthermore, the disclosed dynamic routing system can be advantageously tightened or loosened according to predicted market demand and geographic dispersion (as described above).

The demand distribution may be modeled differently. The demand distribution may be modeled advantageously according to population density to better capture density of a market. FIG. 14 illustrates different examples of these models, which include uniform dispersion, random dispersion, and clumped dispersion.

In some embodiments, the system uses zip code centroids as means of mapping. A constant value (e.g., associated with time between zip code centroids) or a variable value (depending on exact locations within a zip code, advantageously accounting for different zip code sizes) can be used as mapping estimate between two addresses in a same zip code.

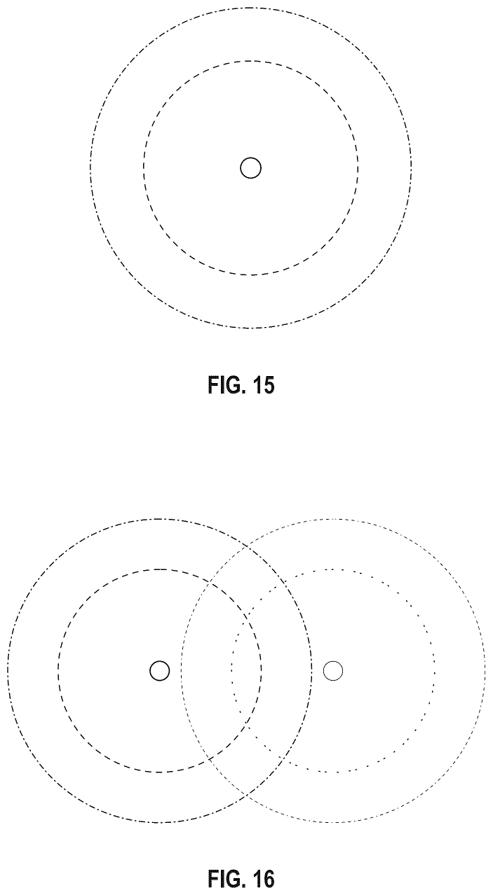

FIGS. 15-17B illustrate an example of determining reachability ellipses (e.g., determining slack) for optimizing the healthcare professional's itinerary described herein. FIG. 15 shows one booked visit and rings associated with one buffer. FIG. 16 shows a second booked visit and rings associated with one buffer, overlaid onto the representations from FIG. 15.

For example, the maximum continuous travel duration can be 50 minutes, the two booked visits are 85 minutes away, and the visits are each 30 minutes long. Based on this information, the number of buffer would be two because floor (2*85/50)−1=3−1=2 slots, and the slack would be 210 minutes because slack=2 slots*(30 minute visits+50 minute travel)+50 minute travel.

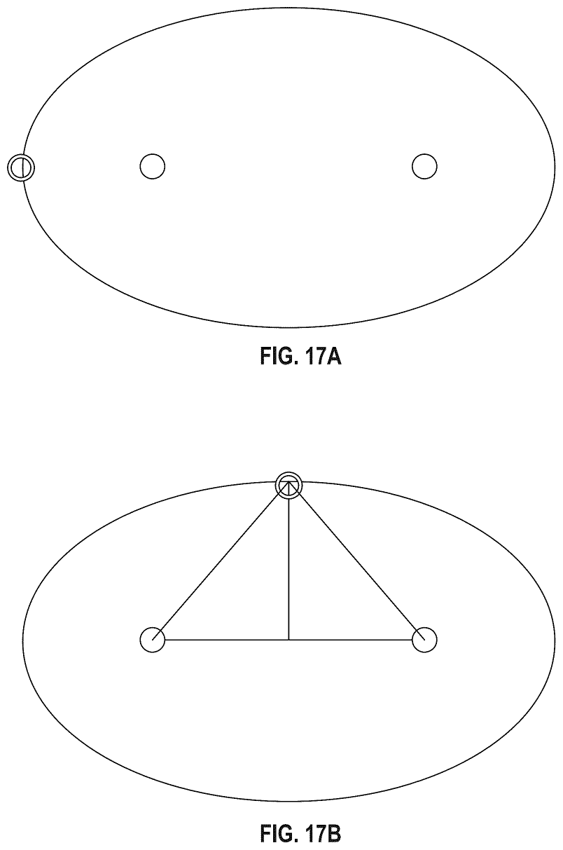

As described above, the slack would affect the width of the ellipse. Referring back to the pin and thread representation, at the far left or far right perimeter, the thread would equal to the travel duration between the visits in addition to 2 times the length between either edge and the visit. Subtracting the slack from the buffered duration thus only leaves us a certain amount of string remaining. That number divided by 2 is the location of the left edge or right edge. Additionally, because the width accounting for both sides is created, that distance from the right edge or left edge can be taken, multiplied it by 2, and added back the distance between the two visits, and arrive at the travel duration. This is illustrated in FIG. 17A and determined below.
Distance between visits=85 minutes
210 minutes of slack−85 travel minutes=125 minutes
Distance from left visit to left edge=125 minutes/2
Distance from right visit to right edge=125 minutes/2
Width=Left Edge+Right Edge+Distance between visits
Width=2*(Slack−Distance)/2+Distance
Width=Slack-Distance+Distance
Width=Slack

To determine the height of the ellipse, the middle point between the two booked visit would form two right triangles, as illustrated in FIG. 17B. Using Pythagorean Theorem, the height of the ellipse can be determined as follows.
a²+b²=c²
a=85/2
c=210/2
b=sqrt((210/2)²−(85/2)²)=96.014

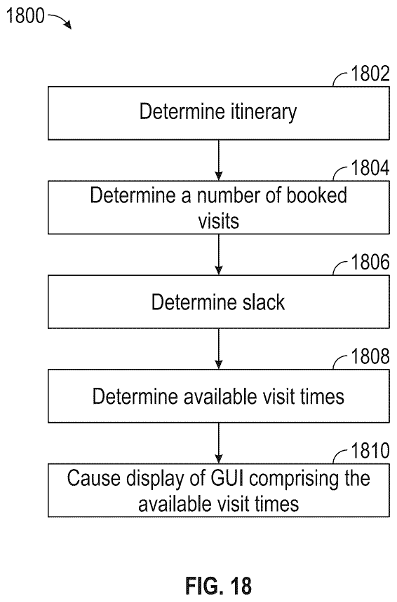

FIG. 18 illustrates an exemplary method 1800 for care provider visit management, in accordance with some embodiments. In some embodiments, the steps of method 1800 are performed by one or more components described with respect to FIGS. 1-17B, and/or components of system 1900. It should be appreciated that steps described with respect to FIG. 18 are exemplary. The method 1800 may include fewer steps, additional steps, or different order of steps than described. Additional examples of method 1800 are described with respect to FIGS. 1-17B, and it is appreciated that the steps of method 1800 leverage the features and advantages described with respect to these Figures.

In some embodiments, the method 1800 comprises determining an itinerary of a healthcare professional (step 1802). For example, as described with respect to FIGS. 1-17B, a healthcare professional's itinerary is determined (e.g., based on information received by the system).

In some embodiments, the method 1800 comprises determining, based on the itinerary, a number of booked visits for the healthcare professional (step 1804). For example, as described with respect to FIGS. 1-17B, based on the healthcare professional's itinerary, a number of booked visits (e.g., zero, one, two or more) is determined.

In some embodiments, the method 1800 comprises determining, based on the itinerary, slack associated with the healthcare professional (step 1806). For example, as described with respect to FIGS. 1-17B, based on the healthcare professional's itinerary, slack is determined.

In some embodiments, the method 1800 comprises determining, based on the slack, one or more available visit times for the healthcare professional (step 1808). For example, as described with respect to FIGS. 1-17B, based on the determined slack, available visit times for the healthcare professional is determined.

In some embodiments, the method 1800 comprises causing display of a graphical user interface (GUI) comprising the one or more available visit times (step 1810). For example, as described with respect to FIGS. 1-17B, the determined available visit times for the healthcare professional are displayed on a GUI of a device (e.g., a patient's device, a caregiver's device, a provider's device).

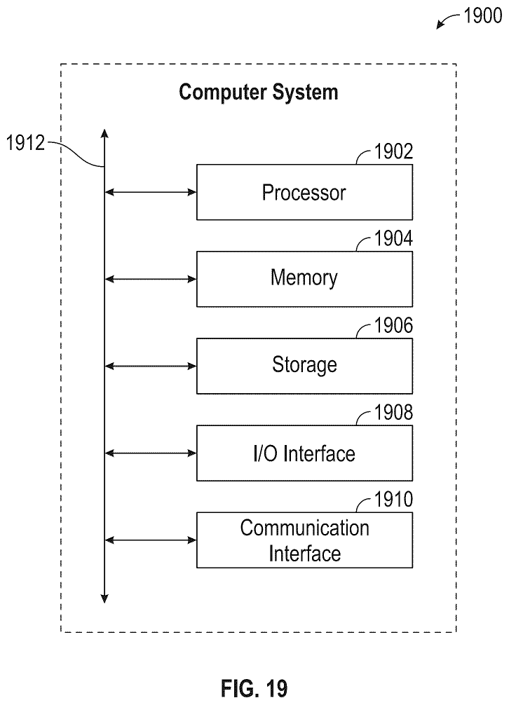

FIG. 19 illustrates an example computer system 1900. In some embodiments, a disclosed system comprises the computer system 1900. In particular embodiments, one or more computer systems 1900 perform one or more steps of one or more methods described or illustrated herein. In particular embodiments, one or more computer systems 1900 provide functionality described or illustrated herein. In particular embodiments, software running on one or more computer systems 1900 performs one or more steps of one or more methods described or illustrated herein or provides functionality described or illustrated herein. Particular embodiments include one or more portions of one or more computer systems 1900. Herein, reference to a computer system may encompass a computing device, and vice versa, where appropriate. Moreover, reference to a computer system may encompass one or more computer systems, where appropriate.

This disclosure contemplates any suitable number of computer systems 1900. This disclosure contemplates computer system 1900 taking any suitable physical form. As example and not by way of limitation, computer system 1900 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1900 may include one or more computer systems 1900; be unitary or distributed; span multiple locations; span multiple machines; span multiple data centers; or reside in a cloud, which may include one or more cloud components in one or more networks. Where appropriate, one or more computer systems 1900 may perform without substantial spatial or temporal limitation one or more steps of one or more methods described or illustrated herein. As an example and not by way of limitation, one or more computer systems 1900 may perform in real time or in batch mode one or more steps of one or more methods described or illustrated herein. One or more computer systems 1900 may perform at different times or at different locations one or more steps of one or more methods described or illustrated herein, where appropriate.

In particular embodiments, computer system 1900 includes a processor 1902, memory 1904, storage 1906, an input/output (I/O) interface 1908, a communication interface 1910, and a bus 1912. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1902 includes hardware for executing instructions, such as those making up a computer program. As an example and not by way of limitation, to execute instructions, processor 1902 may retrieve (or fetch) the instructions from an internal register, an internal cache, memory 1904, or storage 1906; decode and execute them; and then write one or more results to an internal register, an internal cache, memory 1904, or storage 1906. In particular embodiments, processor 1902 may include one or more internal caches for data, instructions, or addresses. This disclosure contemplates processor 1902 including any suitable number of any suitable internal caches, where appropriate. As an example and not by way of limitation, processor 1902 may include one or more instruction caches, one or more data caches, and one or more translation lookaside buffers (TLBs). Instructions in the instruction caches may be copies of instructions in memory 1904 or storage 1906, and the instruction caches may speed up retrieval of those instructions by processor 1902. Data in the data caches may be copies of data in memory 1904 or storage 1906 for instructions executing at processor 1902 to operate on; the results of previous instructions executed at processor 1902 for access by subsequent instructions executing at processor 1902 or for writing to memory 1904 or storage 1906; or other suitable data. The data caches may speed up read or write operations by processor 1902. The TLBs may speed up virtual-address translation for processor 1902. In particular embodiments, processor 1902 may include one or more internal registers for data, instructions, or addresses. This disclosure contemplates processor 1902 including any suitable number of any suitable internal registers, where appropriate. Where appropriate, processor 1902 may include one or more arithmetic logic units (ALUs); be a multi-core processor; or include one or more processors 1902. Although this disclosure describes and illustrates a particular processor, this disclosure contemplates any suitable processor.

In particular embodiments, memory 1904 includes main memory for storing instructions for processor 1902 to execute or data for processor 1902 to operate on. As an example and not by way of limitation, computer system 1900 may load instructions from storage 1906 or another source (such as, for example, another computer system 1900) to memory 1904. Processor 1902 may then load the instructions from memory 1904 to an internal register or internal cache. To execute the instructions, processor 1902 may retrieve the instructions from the internal register or internal cache and decode them. During or after execution of the instructions, processor 1902 may write one or more results (which may be intermediate or final results) to the internal register or internal cache. Processor 1902 may then write one or more of those results to memory 1904. In particular embodiments, processor 1902 executes only instructions in one or more internal registers or internal caches or in memory 1904 (as opposed to storage 1906 or elsewhere) and operates only on data in one or more internal registers or internal caches or in memory 1904 (as opposed to storage 1906 or elsewhere). One or more memory buses (which may each include an address bus and a data bus) may couple processor 1902 to memory 1904. Bus 1912 may include one or more memory buses, as described below. In particular embodiments, one or more memory management units (MMUs) reside between processor 1902 and memory 1904 and facilitate accesses to memory 1904 requested by processor 1902. In particular embodiments, memory 1904 includes random access memory (RAM). This RAM may be volatile memory, where appropriate, this RAM may be dynamic RAM (DRAM) or static RAM (SRAM). Moreover, where appropriate, this RAM may be single-ported or multi-ported RAM. This disclosure contemplates any suitable RAM. Memory 1904 may include one or more memories 1904, where appropriate. Although this disclosure describes and illustrates particular memory, this disclosure contemplates any suitable memory.

In particular embodiments, storage 1906 includes mass storage for data or instructions. As an example and not by way of limitation, storage 1906 may include a hard disk drive (HDD), a floppy disk drive, flash memory, an optical disc, a magneto-optical disc, magnetic tape, or a Universal Serial Bus (USB) drive or a combination of two or more of these. Storage 1906 may include removable or non-removable (or fixed) media, where appropriate. Storage 1906 may be internal or external to computer system 1900, where appropriate. In particular embodiments, storage 1906 is non-volatile, solid-state memory. In particular embodiments, storage 1906 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1906 taking any suitable physical form. Storage 1906 may include one or more storage control units facilitating communication between processor 1902 and storage 1906, where appropriate. Where appropriate, storage 1906 may include one or more storages 1906. Although this disclosure describes and illustrates particular storage, this disclosure contemplates any suitable storage.

In particular embodiments, I/O interface 1908 includes hardware, software, or both, providing one or more interfaces for communication between computer system 1900 and one or more I/O devices. Computer system 1900 may include one or more of these I/O devices, where appropriate. One or more of these I/O devices may enable communication between a person and computer system 1900. As an example and not by way of limitation, an I/O device may include a keyboard, keypad, microphone, monitor, display, mouse, printer, scanner, speaker, still camera, stylus, tablet, touch screen, trackball, video camera, sensors, magnetic detectors, another suitable I/O device or a combination of two or more of these. An I/O device may include one or more sensors. This disclosure contemplates any suitable I/O devices and any suitable I/O interfaces 1908 for them. Where appropriate, I/O interface 1908 may include one or more device or software drivers enabling processor 1902 to drive one or more of these I/O devices. I/O interface 1908 may include one or more I/O interfaces 1908, where appropriate. Although this disclosure describes and illustrates a particular I/O interface, this disclosure contemplates any suitable I/O interface. The one or more I/O devices may be configured to display a graphical user interface for selection of available visit times.

In particular embodiments, communication interface 1910 includes hardware, software, or both providing one or more interfaces for communication (such as, for example, packet-based communication) between computer system 1900 and one or more other computer systems 1900 or one or more networks. As an example and not by way of limitation, communication interface 1910 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1910 for it. As an example and not by way of limitation, computer system 1900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1900 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1900 may include any suitable communication interface 1910 for any of these networks, where appropriate. Communication interface 1910 may include one or more communication interfaces 1910, where appropriate. Although this disclosure describes and illustrates a particular communication interface, this disclosure contemplates any suitable communication interface.

In particular embodiments, bus 1912 includes hardware, software, or both coupling components of computer system 1900 to each other. As an example and not by way of limitation, bus 1912 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1912 may include one or more buses 1912, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

In some embodiments, a non-transitory computer readable storage medium stores one or more programs, and the one or more programs includes instructions. When the instructions are executed by an electronic device (e.g., computer system 1900) with one or more processors and memory, the instructions cause the electronic device to perform the methods described with respect to FIGS. 1-18.

Those skilled in the art will recognize that the systems described herein are representative, and deviations from the explicilty disclosed embodiments are within the scope of the disclosure.

Although the disclosed embodiments have been fully described with reference to the accompanying drawings, it is to be noted that various changes and modifications will become apparent to those skilled in the art. Such changes and modifications are to be understood as being included within the scope of the disclosed embodiments as defined by the appended claims.

The terminology used in the description of the various described embodiments herein is for the purpose of describing particular embodiments only and is not intended to be limiting. As used in the description of the various described embodiments and the appended claims, the singular forms “a”, “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

1. A method, comprising:
determining an itinerary associated with a healthcare resource;
determining, based on the itinerary, a number of booked visits associated with the healthcare resource;
determining, based on the itinerary, slack associated with the healthcare resource;
determining, based on the slack, one or more available visit times for the healthcare resource; and
causing display of a graphical user interface comprising the one or more available visit times.

2. A system comprising:
a display; and
one or more processors configured to perform a method comprising:
determining an itinerary associated with a healthcare resource;
determining, based on the itinerary, a number of booked visits associated with the healthcare resource;
determining, based on the itinerary, slack associated with the healthcare resource;
determining, based on the slack, one or more available visit times for the healthcare resource; and
causing the display to present a graphical user interface comprising the one or more available visit times.

3. A non-transitory computer-readable medium storing instructions that, when executed by one or more processors, cause the one or more processors to perform a method comprising:
determining an itinerary associated with a healthcare resource;
determining, based on the itinerary, a number of booked visits associated with a healthcare resource;
determining, based on the itinerary, slack associated with the healthcare resource;
determining, based on the slack, one or more available visit times for the healthcare resource; and
causing display of a graphical user interface comprising the one or more available visit times.
